(19) United States
(12) Patent Application Publication
(10) Pub. No.: US 2015/0342656 A1
(43) Pub. Date: Dec. 3, 2015

(54) FRACTURE REDUCTION STRUCTURE

(71) Applicant: Empire Technology Development LLC, Wilmington, DE (US)

(72) Inventor: Nicky Bertollo, Wollstonecraft (AU)

(73) Assignee: Empire Technology Development LLC, Wilmington, DE (US)

(21) Appl. No.: 14/426,161

(22) PCT Filed: Jan. 21, 2014
(86) PCT No.: PCT/US14/12394

§ 371 Date: Mar. 5, 2015

(51) Int. Cl. A61B 17/86; A61B 17/84

(52) U.S. Cl. CPC A61B 17/8685; A61B 17/866; A61B 17/864; A61B 17/863; A61B 17/842; A61B 2017/681

(57) ABSTRACT

Technologies are generally provided for an attachment device to achieve pelvic fracture reduction and stabilization. An example attachment device may include two or more screws coupled together in succession to generate a reduction force when inserted through bony tissue. Each screw may have a hollow center, and a guide wire defining a linear or curvilinear path through bony tissue may be configured to pass through the center of the screws. Successive screw segments may exhibit increasing thread pitch to permit generation of a reduction force across multiple fracture planes. Each screw segment may vary in length, diameter, and pitch to enable customization of a configuration of the attachment device according to anatomical needs. Distal and proximal ends of each screw segment may be configured such that multiple rotational degrees of freedom of successive screws may be permitted about axes forming tangents with a longitudinal axis of the guide wire.

### BACKGROUND

Unless otherwise indicated herein, the materials described in this section are not prior art to the claims in this application and are not admitted to be prior art by inclusion in this section.

Pelvic fractures are among the most serious injuries currently treated by orthopedic surgeons. Common methods of surgical treatment to repair pelvic and acetabular fractures include the use of surgical screws and interfragmentary wire to achieve internal and external fixation. One or more surgical screws may be threaded into the bone on either side of the fracture, and the one or more surgical screws may be connected to rods outside of the skin, forming a frame. Polymethylmethacrylate may also be used to affix the surgical screws to the fracture location and to remodel any bone that has been lost, while the interfragmentary wire may be used for fixation of both the bone and the surgical screws. The pelvis involves multiple bony structures and fracture planes, and it can be difficult to achieve successful fixation with external and internal fixation techniques for complex fractures across multiple fracture planes.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

According to some examples, the present disclosure describes an attachment device to achieve fracture reduction. The attachment device may include a plurality of screws configured to couple together in a chained manner to form an attachment structure for connecting one or more of: hard body tissue and soft body tissue. The attachment device may also include a guide wire configured to be formed into a curved shape.

According to other examples, the present disclosure also describes a method of forming an attachment device to achieve pelvic fracture reduction. The method may include providing a guide wire, wherein the guide wire is configured to be curved to follow multiple fracture planes in a fractured pelvis. The method may also include inserting a plurality of screws, individually and sequentially, over the guide wire along a curvature of the guide wire such that each screw in the plurality of screws forms a segment in an attachment structure for connecting one or more of: hard body tissue and soft body tissue.

According to further examples, the present disclosure also describes a system to achieve pelvic fracture reduction. The system may include a guide wire configured to be formed into a curved shape to follow multiple fracture planes in a fractured pelvis. The system may also include a plurality of screws configured to couple together in a chained manner to form an attachment structure following the curved shape of the guide wire for connecting one or more of: hard body tissue and soft body tissue.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example screw attachment structure inserted in a fractured pelvis;

FIG. 2 illustrates an example screw including a distinct thread pitch and hollow center;

FIG. 3 illustrates example engaging surfaces on screw segments;

FIG. 4 illustrates an example interlocking mechanism for coupling screw segments;

FIG. 5 illustrates example screw segments with varying thread pitches coupled together over a guide wire; and

FIG. 6 illustrates example non-threaded segments coupled with threaded segments over a guide wire;

all arranged in accordance with at least some embodiments as described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, apparatus, systems, and/or devices related to achieving pelvic fracture reduction.

Briefly stated, technologies are generally provided for an attachment device to achieve pelvic fracture reduction and stabilization. An example attachment device may include two or more screws coupled together in succession to generate a reduction force when inserted through bony tissue. Each screw may have a hollow center, and a guide wire defining a linear or curvilinear path through bony tissue may be configured to pass through the center of the screws. Successive screw segments may exhibit increasing thread pitch to permit generation of a reduction force across multiple fracture planes. Each screw segment may vary in length, diameter, and pitch to enable customization of a configuration of the attachment device according to anatomical needs. Distal and proximal ends of each screw segment may be configured such that multiple rotational degrees of freedom of successive screws may be permitted about axes forming tangents with a longitudinal axis of the guide wire.

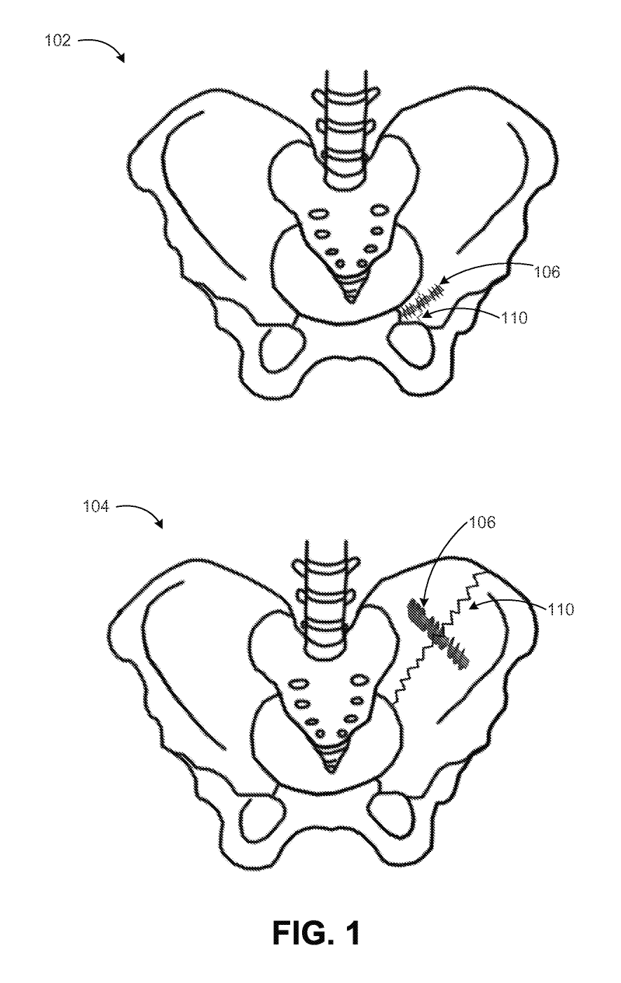

FIG. 1 illustrates an example screw attachment structure inserted in a fractured pelvis, arranged in accordance with at least some embodiments as described herein.

Pelvises 102 and 104 depicted in FIG. 1 demonstrate example fractures 110 that may occur at various locations of the pelvis due to external forces, and other causes. The pelvis consists of the ilium (i.e., iliac wings), ischium, and pubis, which form an anatomic pelvic ring with respect to a sacrum, which is a large, triangular bone at the base of the spine and at the upper and back part of the pelvic cavity. Pelvic fractures 110 may occur as result of high-energy injuries, and characteristics of pelvic fracture can include severe pain, pelvic bone instability, and internal bleeding. Pelvic fractures 110 resulting in bone instability may be treated employing internal fixation, where screws 106 and/or plates may be applied directly onto the fracture site. The screws 106 may apply a reduction force to align fractured bone segments and to stabilize the pelvic bones at the site of the fracture. Due to the nature of the pelvic ring including multiple bony structures with varying bone shapes, and the fact that a fracture may include multiple bone segments, alignment of fractured bone segments, stabilization and fixation at the fracture site can be difficult using fixation methods such as straight bone screws having cancellous or cortical threads. In other embodiments, the screws may be designed with screw threads to apply a distraction force to bone.

A system according to embodiments may improve some complications of pelvic fracture repair through the utilization of surgical screws 106 having differing pitches, diameters, and lengths. Additionally, a system according to embodiments may provide surgical screws 106 configured to be connected together in series to allow for the engaged screws 106 to achieve a curved path through the bone and to permit multiple rotational degrees of freedom and stabilization across multiple fracture planes.

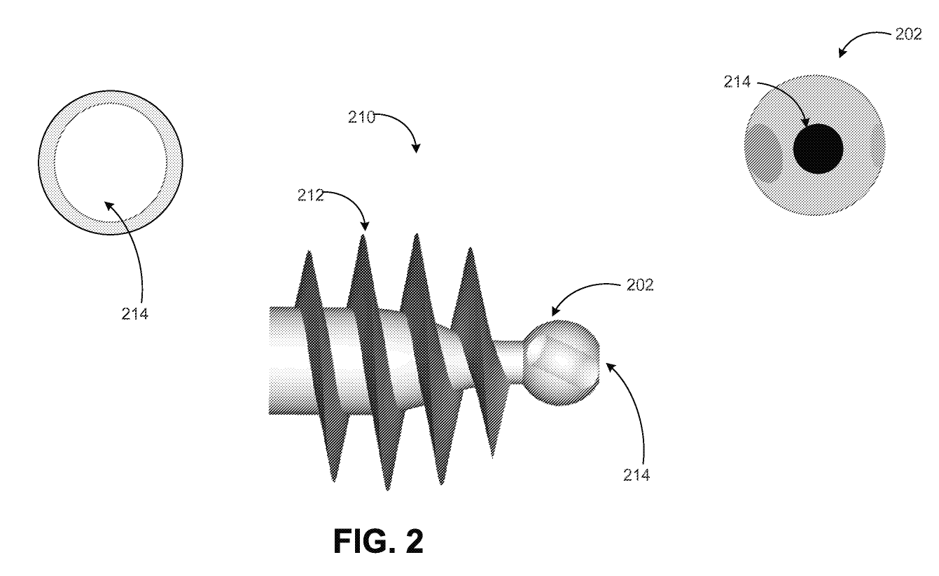

FIG. 2 illustrates an example screw including a distinct thread pitch and hollow center, arranged in accordance with at least some embodiments as described herein.

In a system according to embodiments, an attachment device may be provided to achieve fracture reduction at a fracture site in bony tissue, such as a pelvis. The attachment device may include at least one screw segment 210 configured to be connected in series with at least one additional screw segment in order to achieve a linear or curvilinear path through the bony tissue and soft body tissue, such as muscle and fascia. The ability to combine two or more screw segments in succession may provide the ability to customize an overall length of the attachment device, which may remove the need for large inventories of different screw lengths and designs. For example, combining two or more screws in succession may provide the opportunity to design a combination of screw thread pitch, diameter, overall length, and curvature to meet the needs of a patient's anatomy and a fracture site.

In an example embodiment, each screw segment 210 may be threaded 212 with a distinct thread pitch to enable the screw 210 to be screwed into hard and soft tissue. A diameter of the screw may be a constant diameter, and in some embodiments, the diameter may be tapered from a proximal end to a distal end to aid in insertion of the screw through self-tapping. The diameter may also exhibit a radial profile such that proximal and distal ends may have a narrower diameter than a middle portion of the screw. This feature is incorporated to facilitate implantation of the device along a curved path and to ensure thread contact along this path. Additionally, each screw 210 in the series of screws may have a hollow center to enable a guide wire to fit through the hollow center 214 of each screw to direct the curvilinear or linear path through the bony tissue.

Example screws may be composed from one or more of a biodegradable material, a metal material, a composite material, and a polymer material. Additionally, the guide wire may also be composed from similar materials such as a biodegradable material, a metal material, a composite material, and a polymer material. The attachment device may be composed of two or more different materials concurrently to produce a hybrid type of attachment device. For example, distal screw segments may be composed from a resorbable material, such as a polymeric material, such that they may be resorbed by the body, and proximal screw segments may be non-resorbable and removed when necessary. The guide wire may be removed after insertion of the series of screws have been inserted and positioned. In some scenarios, the guide wire may remain in situ, and the guide wire may also be a resorbable polymeric material such that the guide wire may be resorbed after a period of time.

In an example embodiment, the proximal and distal ends of successive screw segments may be configured to engage via an interlocking mechanism to enable multiple screws to be attached together in succession. An example interlocking mechanism may include a ball and socket mechanism, where a proximal end of a first screw may be a substantially spherical shape 202 configured to engage with a distal end of an adjacent screw, where the distal end of the adjacent screw may be configured as a socket chamber. The hollow center 214 of the screw 210 may extend from the distal end through the proximal end of the screw 210 having the substantially spherical shape 202 to enable the guide wire to pass through the hollow center 214 of the screw 210.

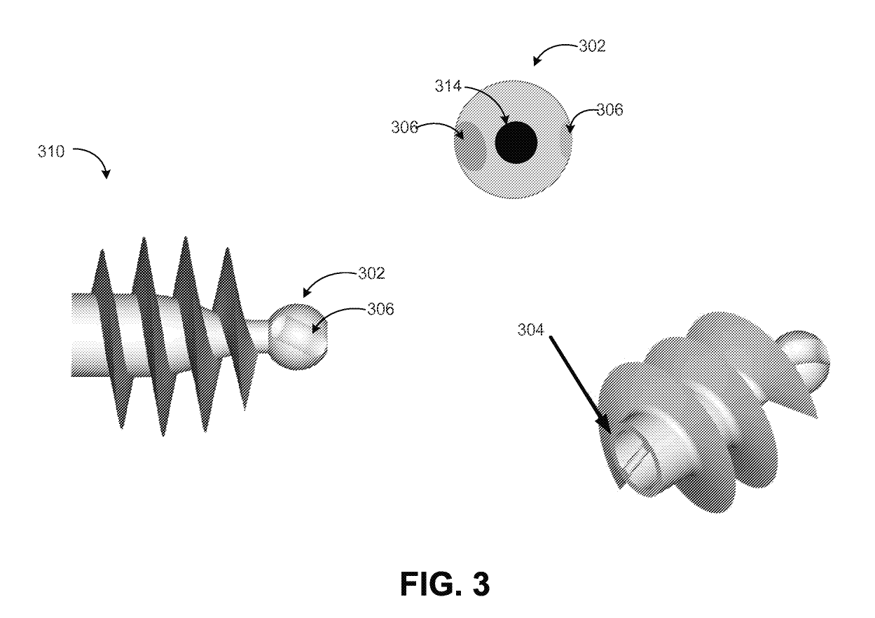

FIG. 3 illustrates example engaging surfaces on screw segments, arranged in accordance with at least some embodiments as described herein.

As briefly described above, two or more screw segments may be configured to be connected together in series to form a fixation or an attachment device for achieving fracture reduction and stabilization. The distal and proximal ends of successive screw segments may be configured to engage via an interlocking mechanism in a manner to permit relative rotation of successive screw segments about individual axes tangential to the centroidal curve of the guide wire. In the absence of a guide wire the screw segments rotate about axes tangential to the centroidal curve of a pre-defined hole. An interlocking mechanism may include a ball and socket mechanism. For example, a proximal end of a first screw may be a substantially spherical shape 302 configured to interlock with a distal end of an adjacent screw, where the distal end of the adjacent screw may be configured as a socket chamber 304. The substantially spherical shape 302 may include an engaging mechanism 306, such as a channel, to enable interlocking with the chamber 304 to resist a tensile axial force.

Additionally, each of the screw segments may include an interface to enable the screw segment to be engaged with a driver for connecting and advancing the screw segments. An interface for engagement with a driver may include a one or more recesses configured to engage with the driver.

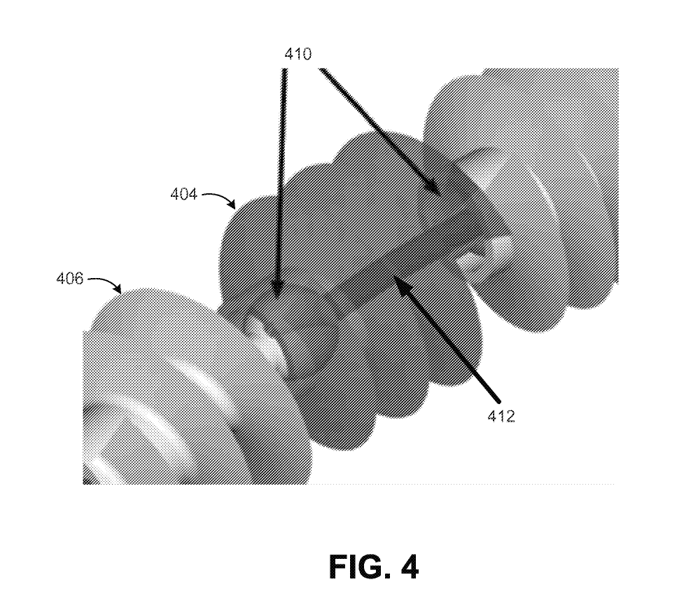

FIG. 4 illustrates an example interlocking mechanism for coupling screw segments, arranged in accordance with at least some embodiments as described herein.

As described above in conjunction with FIG. 3, proximal and distal ends of each individual screw segment may exhibit an interlocking mechanism 410 which, when arranged in a serial configuration and engaged, may permit multiple rotational degrees of freedom such as that which would be afforded by a universal joint. The interlocking mechanism 410 may include a substantially spherical shaped proximal end of a first screw 406 configured to fit or engage within a chamber distal end of a second screw 404. When the spherical shaped proximal end of the first screw 410 is engaged with the chamber distal end of the second screw 410, the engaged ends may function as a universal joint to enable two or more screws engaged in succession to follow and maintain a curved path, such as that dictated by the guide wire, as well as a straight path when needed.

Additionally, interlocking of successive segments could be achieved employing an interlocking mechanism to secure each segment and to prevent the individual screw segments from backing out through counter-rotation. The interlocking mechanism may also enable a counter-torque to be applied to the full length of the series of connected screws, which may promote the application of a torque to facilitate removal of the device in a subsequent surgical procedure. The interlocking mechanism may be applied selectively to particular segments as desired, through selection of screw segments exhibiting the interlocking mechanism, or through an activation of the interlocking mechanism in situ.

In a system according to embodiments, a guide pin or guide wire 412 may define a desired linear or curvilinear path of the attachment device, and each screw may be inserted, individually and sequentially, over the guide wire 412 along the defined path of the guide wire 412 such that each screw in the plurality of screws forms a segment in the attachment device. The proximal and distal ends of adjacent screws in the attachment structure may be engaged by inserting the spherical shaped proximal end within the chamber distal end and rotating the proximal end to engage the spherical shaped end with the chamber end. When the proximal and distal ends are engaged, rotation of successive screw segments about axes tangential to a centroidal curve of the guide wire 412 may be permissible. Additionally, the engaged proximal and distal ends acting as pseudo universal joints between adjacent screw segments may also permit transmission of a driving torque. Furthermore, the engagement of the proximal and distal ends through relative rotation may provide axial tensile force resistance, thereby allowing generation of a reduction force across one or more fracture planes.

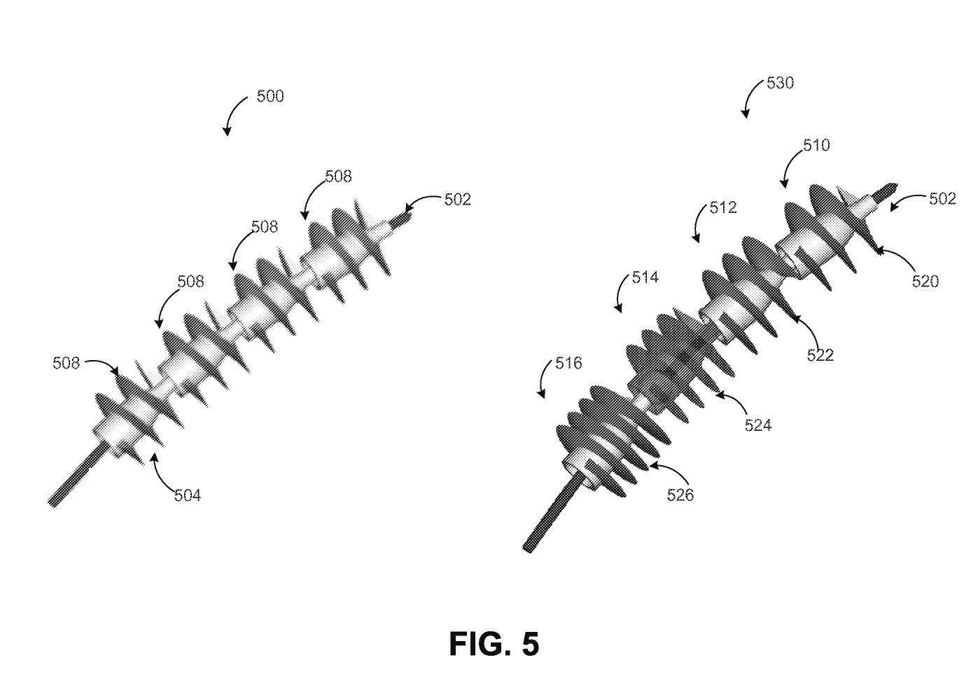

FIG. 5 illustrates example screw segments with varying thread pitches coupled together over a guide wire, arranged in accordance with at least some embodiments as described herein.

In a system according to embodiments, two or more screws (510, 512, 514, 516) may be coupled together sequentially such that each screw may form a segment in an attachment device (500, 520) to fixate bony and soft tissue, and to apply a reduction force across one or more fracture planes. In some example embodiments, as illustrated by attachment device 500, two or more screws 504 may be guided over a guide wire 502 to follow a linear or curvilinear path defined by the guide wire. Proximal and distal ends of adjacent screws may be engaged to enable rotation of adjacent screws about the guide wire 502. In an example embodiment, each successive screw may exhibit equal thread pitches 508. A surgeon may maintain a reduction force across the attachment device 500 including two or more successive screws having equal thread pitches 508 employing clamps, for example to apply the reduction force.

In another example embodiment, as illustrated by attachment device 530, successive screws may exhibit increasing thread pitches (520, 522, 524, 526), which may result in a generation of a reduction force by virtue of a differential in relative advancement through bony segments. For example, a leading screw 510 may have a wider or courser thread pitch 520 than an adjacent screw 512 such that the reduction force may be generated by increased relative advancement with each successive screw. Additionally, the thread pitch 520 of the leading screw 510 may be tapered to facilitate self-tapping for advancing the attachment device through bony tissue.

In an alternative embodiment, a thread pitch of each successive screw may be modulated along the length of the path of the guide wire 502 in accordance with surgical objectives and bone quality. Additionally, a thread pitch of a screw may be modulated along a length of each individual screw in a linear or non-linear manner.

In a system according to embodiments, a length, diameter and thread pitch of successive screws may be varied such that a multitude of combinations of attachment devices to achieve fixation of fractured bone may be achieved. By combining different lengths, diameters, and thread pitches, natural bony geometry and architecture of a fracture site across multiple fracture planes can be accommodated, and may increase the ability to stability and fixate complex fractures.

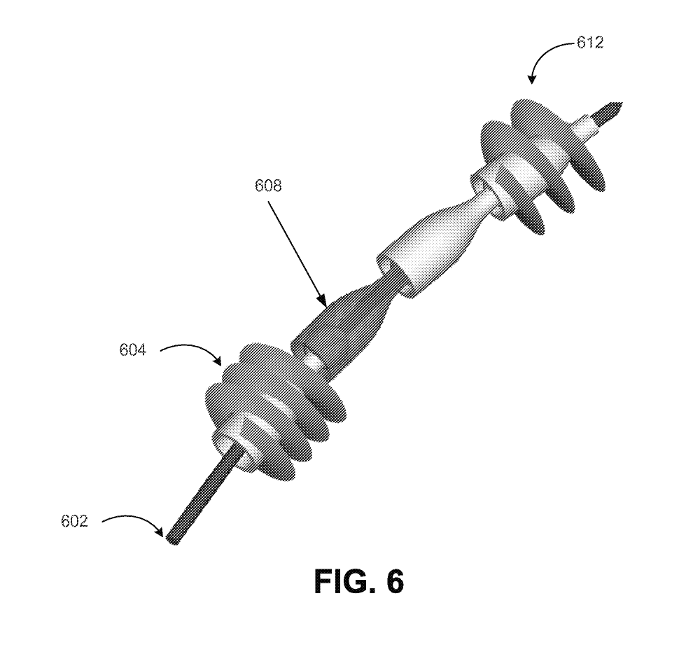

FIG. 6 illustrates example non-threaded segments coupled with threaded segments over a guide wire, arranged in accordance with at least some embodiments as described herein.

In an additional embodiment, unthreaded, or blank, screw segments may also be incorporated with a series of screw segments to form the attachment devices where a reduction force or bone integration may not be necessary. For example, an unthreaded screw 608 may be coupled with a threaded leading screw 612 and a threaded distal screw 604 over a guide wire to produce an attachment device with threaded proximal and distal ends. Such an attachment device may be employed for fracture fixation in long bones where a reduction force may not need to be applied to at least a portion of the bone. The leading screw 612 may have a wider thread pitch than the distal screw 602 in order to produce a reduction force as the screw attachment structure is inserted within bony tissue.

In a system according to embodiments, the screw attachment structure to be inserted in a body for reduction of complex fractures may be assembled intra-operatively in situ. For example, the screw attachment structure may be implanted one screw segment at a time, and each segment may connect and engage with a successive segment after placement within bony tissue in the body. In an example scenario, a lead screw segment may be inserted into a final preferred position within the bony tissue. The guide wire may be inserted within the lead screw segment to direct a desired path of the screw attachment structure. A successive screw segment may be inserted over the guide wire and may engage with the lead screw segment. Similarly, additional successive screw segments may be inserted over the guide wire, such that each screw segment may be positioned in place in situ and may engage with a preceding screw segment until all of the screw segments comprising the screw attachment structure are in place.

In a further embodiment, the screw attachment structure for insertion in a body for reduction of complex fractures may be provided as a pre-assembled unit. For example, two or more screw segments as described above, may be coupled together over the guide wire pre-operatively in a desired arrangement with a preferred curvature. The guide wire may be a pliable material to enable the path and curvature of the guide wire and the screw segments to be adjusted intra-operatively to suit a surgical objective, such as the reduction of a complex fracture. Additionally, the guide wire may be composed from two or more segments to facilitate manipulation of the curvature and path. In such an embodiment, a proximal and distal end of the guide wire may be configured to constrain the first and last screw segments, and may also extend beyond the screw segment to enable manual manipulation by a surgeon for adjusting the position, orientation, and curvature of the screw attachment structure in situ. Additionally, a driver may be used to apply torque to the screw segments while concurrently enabling grasping and locking the central guide wire to limit rotation of the screw attachment structure.

While embodiments have been discussed above using specific examples, components, and configurations, they are intended to provide a general guideline to be used to achieve complex fracture reduction in a pelvis. Fracture reduction may be achieved in other areas of the body, as well, such as a clavicle, or other areas where complex fractures may occur and may necessitate fixation and reduction over a curved path. Furthermore, a screw attachment structure configured to follow a curved path may be employed in applications where anchorage in bone may be required, such as when using sutures for other surgical applications where the sutures may be anchored to a structure anchored in bone. These examples do not constitute a limitation on the embodiments, which may be implemented using other components, modules, and configurations using the principles described herein. Furthermore, actions discussed above may be performed in various orders, especially in an interlaced fashion.

According to some examples, the present disclosure describes an attachment device to achieve fracture reduction. The attachment device may include a plurality of screws configured to couple together in a chained manner to form an attachment structure for connecting one or more of hard body tissue and soft body tissue. The attachment device may also include a guide wire configured to be formed into a curved shape.

According to some examples, the attachment device may include each screw in the plurality of screws having a hollow center. The attachment device may further include the guide wire being configured to pass through the hollow center of each screw in the plurality of screws. The attachment device may further be configured where the plurality of screws share a substantially equal thread pitch.

According to some examples, the attachment device may further include each screw in the plurality of screws having a distinct thread pitch. The attachment device may further include a pitch on each screw in the plurality of screws being configured to vary along a length of the screw. The attachment device may further include a proximal end of each screw in the plurality of screws being configured to be coupled with a distal end of another screw via an interlocking mechanism.

According to further examples, the attachment device may further include a first screw and a second screw being configured to rotate about a tangential axis to a centroidal curve of the guide wire when a proximal end of the first screw is coupled with a distal end of the second screw. The attachment device may further include a first screw and a second screw being configured to be in a fixed position in relation to each other when a proximal end of the first screw is coupled with a distal end of the second screw.

According to some examples, the attachment device may further include the interlocking mechanism including a ball and socket mechanism. The interlocking mechanism may be configured to secure each segment to prevent each screw from backing out through counter-rotation, and wherein the interlocking mechanism is configured to be activated in situ. The attachment device may further include the distal end of the second screw being a socket chamber and the proximal end of a first screw being a substantially spherical head configured to fit within the socket chamber of the second screw and to be rotated to lock into place within the socket chamber.

According to further examples, the attachment device may further include the guide wire being composed from one or more of: a biodegradable material, a metal material, a composite material, and a polymer material. The attachment device may further include the plurality of screws being composed from one or more of: a biodegradable material, a metal material, a composite material, and a polymer material.

According to some examples, the attachment device may further include each screw in the plurality of screws being substantially straight. The attachment device may further include each screw in the plurality of screws being curved at a predefined radius of curvature. The attachment device may further include the plurality of screws having substantially the same diameter.

According to some examples, the attachment device may further include each screw in the plurality of screws having a different diameter. The attachment device may further include a diameter of each screw in the plurality of screws being tapered from a proximal end to a distal end. The attachment device may further include at least one screw in the plurality of screws being not threaded.

According to some examples, the present disclosure describes a method of forming an attachment device to achieve pelvic fracture reduction. The method may include providing a guide wire, where the guide wire is configured to be curved to follow multiple fracture planes in a fractured pelvis. The method may additionally include inserting a plurality of screws, individually and sequentially, over the guide wire along a curvature of the guide wire such that each screw in the plurality of screws forms a segment in an attachment structure for connecting one or more of: hard body tissue and soft body tissue.

According to some examples, the method may further comprise coupling the plurality of screws together in a chained manner to form the attachment structure. According to additional examples, the method may further comprise configuring each screw in the plurality of screws to have a hollow center.

According to some examples, the method may further comprise the guide wire being configured to pass through the hollow center of each screw in the plurality of screws.

According to further examples, the method may further comprise threading each screw in the plurality of screws with a substantially equal thread pitch. The method may additionally comprise threading each screw in the plurality of screws with a distinct thread pitch. The method may further comprise varying a pitch on each screw in the plurality of screws along a length of the screw.

According to additional examples, the method may further comprise coupling a proximal end of each screw in the plurality of screws with a distal end of another screw via an interlocking mechanism. According to some examples, the method may further comprise configuring a first screw and a second screw to rotate about a tangential axis to a centroidal curve of the guide wire when a proximal end of the first screw is coupled with a distal end of the second screw.

According to some examples, the method may comprise configuring a first screw and a second screw to be in a fixed position in relation to each other when a proximal end of the first screw is coupled with a distal end of the second screw. The method may additionally comprise the interlocking mechanism including a ball and socket mechanism.

According to some examples, the method may further comprise configuring the distal end of the second screw to be a socket chamber and the proximal end of a first screw to have a substantially spherical head, where the substantially spherical head of the first screw is configured to fit within the socket chamber of the second screw and to be rotated to lock into place within the socket chamber.

According to some examples, the method may further comprise composing the guide wire from one or more of: a biodegradable material, a metal material, a composite material, and a polymer material. According to additional examples, the method may further comprise composing the plurality of screws from one or more of: a biodegradable material, a metal material, a composite material, and a polymer material.

According to additional examples, the method may further comprise configuring each screw in the plurality of screws to be substantially straight. The method may additionally comprise configuring each screw in the plurality of screws to have substantially the same diameter.

According to some examples, the method may further comprise configuring each screw in the plurality of screws to have a different diameter. The method may further comprise tapering a diameter of each screw in the plurality of screws from a proximal end to a distal end. The method may include where at least one screw in the plurality of screws is not threaded.

According to some examples, the present disclosure describes a system to achieve pelvic fracture reduction. The system may include a guide wire configured to be formed into a curved shape to follow multiple fracture planes in a fractured pelvis. The system may further include a plurality of screws configured to couple together in a chained manner to form an attachment structure following the curved shape of the guide wire for connecting one or more of hard body tissue and soft body tissue.

According to some examples, each screw in the plurality of screws may have a hollow center. The system may include the guide wire being configured to pass through the hollow center of each screw in the plurality of screws. The system may include the plurality of screws sharing a substantially equal thread pitch.

According to additional examples, the system may include each screw in the plurality of screws having a distinct thread pitch. The system may include a pitch on each screw in the plurality of screws being configured to vary along a length of the screw. The system may include a proximal end of each screw in the plurality of screws being configured to be coupled with a distal end of another screw via an interlocking mechanism.

According to some examples, the system may include a first screw and a second screw being configured to rotate about a tangential axis to a centroidal curve of the guide wire when a proximal end of the first screw is coupled with a distal end of the second screw. The system may include a first screw and a second screw being configured to be in a fixed position in relation to each other when a proximal end of the first screw is coupled with a distal end of the second screw.

According to additional examples, the interlocking mechanism may include a ball and socket mechanism. According to additional examples, the distal end of the second screw may be a socket chamber and the proximal end of a first screw may be a substantially spherical head configured to fit within the socket chamber of the second screw and to be rotated to lock into place within the socket chamber.

According to some examples, the system may include the guide wire, which may be composed from one or more of: a biodegradable material, a metal material, a composite material, and a polymer material. The system may include the plurality of screws, which may be composed from one or more of: a biodegradable material, a metal material, a composite material, and a polymer material.

According to some examples, the system may include each screw in the plurality of screws, being substantially straight. The system may include the plurality of screws having substantially the same diameter.

According to some examples, each screw in the plurality of screws may have a different diameter. According to some examples, a diameter of each screw in the plurality of screws may be tapered from a proximal end to a distal end. According to different examples, at least one screw in the plurality of screws may not be threaded.

According to further examples, at least one screw in the plurality of screw segments may be configured to engage with a driver through one or more recesses to advance the screw segment. The plurality of screws may be coupled together in a desired arrangement with a predefined curvature to form the attachment structure prior to insertion in the body. The guide wire may be composed from two or more segments to facilitate manipulation of a curvature and a path of the attachment structure.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected,” or “operably “coupled,” to each other to achieve the desired functionality, and any two components capable of being so associated may also be viewed as being “operably couplable,” to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An attachment device to achieve fracture reduction, the attachment device comprising:
a plurality of screws configured to couple together in a chained manner to form an attachment structure for connecting one or more of: hard body tissue and soft body tissue, a proximal end of each screw in the plurality of screws coupled with a distal end of another screw via an interlocking mechanism such that a proximal end of a first screw is configured to fit within a socket chamber comprising a distal end of a second screw; and
a guide wire configured to be formed into a curved shape.

2. (canceled)

3. (canceled)

4. The attachment device of claim 1, wherein the plurality of screws share a substantially equal thread pitch.

5. The attachment device of claim 1, wherein each screw in the plurality of screws has a distinct thread pitch that is configured to vary along a length of each screw.

6.-10. (canceled)

11. The attachment device of claim 1, wherein the interlocking mechanism is configured to secure each segment to prevent each screw from backing out through counter-rotation, and wherein the interlocking mechanism is configured to be activated in situ.

12. The attachment device of claim 1, wherein the proximal end of the first screw is a substantially spherical head configured to be rotated to lock into place within the socket chamber.

13. (canceled)

14. (canceled)

15. The attachment device of claim 1, wherein each screw in the plurality of screws is substantially straight.

16. The attachment device of claim 1, wherein the plurality of screws have substantially the same diameter.

17. The attachment device of claim 1, wherein each screw in the plurality of screws has a different diameter.

18. The attachment device of claim 1, wherein a diameter of each screw in the plurality of screws is tapered from the proximal end to the distal end.

19. The attachment device of claim 1, wherein at least one screw in the plurality of screws is not threaded.

20. A method to form an attachment device to achieve pelvic fracture reduction, comprising:
providing a guide wire, wherein the guide wire is configured to be curved to follow multiple fracture planes in a fractured pelvis; and
inserting a plurality of screws, individually and sequentially, over the guide wire along a curvature of the guide wire such that each screw in the plurality of screws forms a segment in an attachment structure for connecting one or more of: hard body tissue and soft body tissue, wherein a proximal end of each screw in the plurality of screws is coupled with a distal end of another screw via an interlocking mechanism such that a proximal end of a first screw is configured to fit within a socket chamber comprising a distal end of a second screw.

21. (canceled)

22. The method of claim 20, further comprising:
configuring each screw in the plurality of screws to have a hollow center, wherein the guide wire is configured to pass through the hollow center of each screw in the plurality of screws.

23.-27. (canceled)

28. The method of claim 20, further comprising:
configuring the first screw and the second screw to rotate about independent tangential axes to a centroidal curve of the guide wire when the proximal end of the first screw is coupled with the distal end of the second screw.

29. The method of claim 20, further comprising:
configuring the first screw and the second screw to be in a fixed position in relation to each other when the proximal end of the first screw is coupled with the distal end of the second screw.

30.-39. (canceled)

40. A system to achieve pelvic fracture reduction, the system comprising:
a guide wire configured to be formed into a curved shape to follow multiple fracture planes in a fractured pelvis; and
a plurality of screws configured to couple together in a chained manner to form an attachment structure following the curved shape of the guide wire for connecting one or more of:
hard body tissue and soft body tissue, a proximal end of each screw in the plurality of screws coupled with a distal end of another screw via an interlocking mechanism such that a proximal end of a first screw is configured to fit within a socket chamber comprising a distal end of a second screw.

41.-50. (canceled)

51. The system of claim 40, wherein the guide wire is composed from one or more of: a biodegradable material, a metal material, a composite material, and a polymer material.

52. The system of claim 40, wherein the plurality of screws are composed from one or more of: a biodegradable material, a metal material, a composite material, and a polymer material.

53.-58. (canceled)

59. The system of claim 40, wherein at least one screw in the plurality of screws is configured to engage with a driver through one or more recesses to advance a screw segment.

60. The system of claim 40, wherein the plurality of screws are coupled together in a desired arrangement with a predefined curvature to form the attachment structure prior to insertion in the body.

61. The system of claim 40, wherein the guide wire is composed from two or more segments to facilitate manipulation of a curvature and a path of the attachment structure.
